SEQUENCE LISTING (14 sequences)

SEQ ID NO: 1 - protein, 118 aa, Artificial [sequence omitted]

SEQ ID NO: 2 - protein, 108 aa, Artificial [sequence omitted]

SEQ ID NO: 3 - protein, 119 aa, Artificial [sequence omitted]

SEQ ID NO: 4 - protein, 106 aa, Artificial [sequence omitted]

SEQ ID NO: 5 - protein, 119 aa, Artificial [sequence omitted]

SEQ ID NO: 6 - protein, 110 aa, Artificial [sequence omitted]

SEQ ID NO: 7 - protein, 119 aa, Artificial [sequence omitted]

SEQ ID NO: 8 - protein, 106 aa, Artificial [sequence omitted]

SEQ ID NO: 9 - protein, 120 aa, Artificial [sequence omitted]

SEQ ID NO: 10 - protein, 106 aa, Artificial [sequence omitted]

SEQ ID NO: 11 - protein, 213 aa, Artificial
  Synthetic light chain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 450 aa, Artificial
  Synthetic heavy chain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 213 aa, Artificial
  Synthetic light chain
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, Artificial
  Synthetic heavy chain
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0348660 A1
(43) Pub. Date: Nov. 3, 2022

(54) METHODS FOR THE TREATMENT OF ADULT T-CELL LEUKEMIA/LYMPHOMA

(71) Applicants: INSERM (Institut National de la Santé et de la Recherche Médicale), Paris (FR); Université de Paris, Paris (FR); Assistance Publique-Hôpitaux de Paris, Paris (FR); Centre National de la Recherche Scientifique (CNRS), Paris (FR)

(72) Inventors: Vahid ASNAFI, PARIS (FR); Olivier HERMINE, PARIS (FR); Ambroise MARÇAIS, PARIS (FR); Jacques GHYSDAEL, PARIS (FR)

(21) Appl. No.: 17/760,557

(22) PCT Filed: Oct. 1, 2020
(86) PCT No.: PCT/EP2020/077453

§ 371 Date: Mar. 15, 2022

(30) Foreign Application Priority Data
Oct. 2, 2019 (EP) 19306263.5

(51) Int. Cl. C07K 16/28; A61P 35/02

(52) U.S. Cl. CPC C07K 16/2809; A61P 35/02; C07K 2317/24; C07K 2317/515

(57) ABSTRACT

Adult T-cell leukemia/lymphoma (ATL) is an aggressive proliferation of mature activated CD4+ T cells associated with the human T-cell lymphotropic virus type I (HTLV-I). The inventors performed an integrated genomic analysis of a retrospective cohort of 62 ATL patients mainly originating from Africa and the Caribbean area. In particular, they identified a subset of mutations in the TCR/NF-KB pathway (PLCG1, CARD11, PRKCB, CBLB, IRF4, CSNK1A1, FYN, RHOA, VAV1). Furthermore, the inventors investigated the effects of an anti-CD3 antibody (OKT3) exposure on 4 ATL samples including 2 cases harboring CARD 11 and PRKCB gain of function alterations and 2 cases without any TCR pathway mutation. The data suggest that ATL harboring TCR pathway mutations clearly responded to anti-CD3 (FIG. 1B, red+OKT3) and died by apoptosis possibly by a mechanism resembling AICD. Importantly, these TCR-pathway/NFKB mutated patients also showed poorer outcome as compared to unmutated cases. Accordingly, the present invention relates to a method of treating adult T-cell leukemia/lymphoma (ATL) in a patient in need thereof comprising administering to the patient a therapeutically effective amount of an anti-CD3 antibody.

### FIELD OF THE INVENTION

The present invention is in the field of medicine, in particular oncology.

### BACKGROUND OF THE INVENTION

Adult T-cell leukemia/lymphoma (ATL) is an aggressive proliferation of mature activated CD4+ T cells associated with the human T-cell lymphotropic virus type I (HTLV-I). After infection the virus is indeed integrated in the host genome and the viral transactivator protein Tax is expressed and interacts with many cellular pathways that regulate apoptosis, proliferation, DNA repair and epigenetic, resulting in oligoclonal expansions of HTLV-I-infected T cells. These genetically-unstable cells acquire and accumulate genetic abnormalities, resulting in their full transformation. In most of the cases at this stage Tax is no more or poorly or transiently expressed in tumor cells. Interestingly, the genetic/epigenetic alterations found at this terminal stage mainly deregulate the TCR/NF-kB signaling pathway, likely explaining the activated phenotype of these cells (Kataoka K et al (2015) Nat Genet 47:1304-1315). The diversity in clinical features and prognosis of ATL patients has led to its subclassification into smoldering, chronic, lymphoma, and acute subtypes. Patients with aggressive ATL (acute and lymphoma subtypes) generally have a very poor prognosis because of intrinsic chemoresistance of malignant cells, a large tumor burden with multiorgan failure, hypercalcemia, and/or frequent infectious complications due to a profound T-cell immune deficiency. Patients with indolent ATL (ie, the chronic or smoldering subtypes) have a better prognosis. However, data from Japan showed poor long-term survival results when these patients are managed with a watchful-waiting policy until disease progression or with chemotherapy. Indeed, 4-year survival in chronic ATL is less than 30%. Thus there is an unmet medical need for the treatment of ATL.

### SUMMARY OF THE INVENTION

As defined by the claims, the present invention relates to methods for the treatment of adult T-cell leukemia/lymphoma (ATL).

### DETAILED DESCRIPTION OF THE INVENTION

The first object of the present invention relates to a method of treating adult T-cell leukemia/lymphoma (ATL) in a patient in need thereof comprising administering to the patient a therapeutically effective amount of an anti-CD3 antibody.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiments, the patient harbors at least one gain-of-function mutation in a gene involved in the TCR/NF-κB pathway.

As used herein, the term “gain-of-function mutation” refers to any mutation in a gene in which the protein encoded by the gene (i.e. the mutant protein) acquires a function not normally associated with the protein (i.e. the wild type protein) causes or contributes to the lymphoma progression The gain-of-function mutation can be a deletion, addition, or substitution of a nucleotide or nucleotides in the gene which gives rise to the change in the function of the encoded protein.

In the present specification, the name of each gene refers to the internationally recognised name of the corresponding gene, as found in internationally recognised gene sequences and protein sequences databases, including in the database from the HUGO Gene Nomenclature Committee that is available notably at the following Internet address: http://www.gene.ucl.ac.uk/nomenclature/index.html. In the present specification, the name of each of the various genes of interest may also refer to the internationally recognised name of the corresponding gene, as found in the internationally recognised gene sequences and protein sequences database Genbank. Through these internationally recognised sequence databases, the nucleic acid and the amino acid sequences corresponding to each of the gene of interest described herein may be retrieved by the one skilled in the art.

In some embodiments, the patient harbors at least one gain-of-function mutation in PLCG1 (e.g. S345F, Q718K, DelEF730, G869F, S739T, Q916E, E1163K, R48W, D1165H, D1165E, DelDQ1169), CARD11 (e.g. D401N, R179W, R337Q, D401N, R423W, D357V, R377Q, R707C, E626K), PRKCB (e.g. D427N, Q433K, A25V, D470H), CBLB (e.g. InsGH296), IRF4 (e.g. K59R, L70V, L64L, E109Q, S11R), CSNK1A1 (e.g. S189R, L160F), FYN (e.g. T15K, R206C), RHOA (e.g. C16Y, C16R, G17V, G124S, D120N, D120V, A161P, A161V), or VAV1 (e.g. F69V, L145P, R195C, Q498K, M501L, N505T).

As used herein, the term “PLCG1”, for “Phospholipase C, gamma 1”, refers to a gene encoding a protein catalysing the formation of inositol 1,4,5-trisphosphate and diacylglycerol from phosphatidylinositol 4,5-bisphosphate. Its Entrez reference is 5335.

As used herein, the term “CARD11”, for “Caspase Recruitment Domain Family Member 11”, refers to a gene encoding a protein belonging to the membrane-associated guanylate kinase (MAGUK) family, a class of proteins that functions as molecular scaffolds for the assembly of multiprotein complexes at specialized regions of the plasma membrane. Its Entrez reference is 84433.

As used herein, the term “PRKCB”, for “Protein Kinase C Beta”, refers to a gene encoding for a family of serine- and threonine-specific protein kinases that can be activated by calcium and second messenger diacylglycerol. Its Entrez reference is 5579.

As used herein, the term “CBLB”, for “Cbl Prot-Oncogene B”, refers to a gene encoding an E3 ubiquitin-protein ligase which promotes proteosome-mediated protein degradation by transferring ubiquitin from an E2 ubiquitin-conjugating enzyme to a substrate. Its Entrez reference is 868.

As used herein, the term “IRF4”, for “Interferon Regulatory Factor 4”, refers to a gene encoding a transcription factors, characterized by an unique tryptophan pentad repeat DNA-binding domain. Its Entrez reference is 3662.

As used herein, the term “CSNK1A1”, for “Casein Kinase 1 Alpha 1”, refers to a gene encoding a protein casein kinase 1, alpha 1 which has been shown to interact with Centaurin, alpha 1 and AXIN1. Its Entrez reference is 1452.

As used herein, the term “FYN”, also known as “FYN Proto-Oncogene, Src Family Tyrosine Kinase” refers to a member of the protein-tyrosine kinase oncogene family. It encodes a membrane-associated tyrosine kinase that has been implicated in the control of cell growth. Its Entrez reference is 2534.

As used herein, the term “RHOA”, for “Ras Homolog Family Member A”, refers to a gene encoding a member of the Rho family of small GTPases, which cycle between inactive GDP-bound and active GTP-bound states and function as molecular switches in signal transduction cascades. Its Entrez reference is 387.

As used herein, the term “VAV1”, for “Vav Guanine Nucleotide Exchange Factor 1”, refers to a member of the VAV gene family. The VAV proteins are guanine nucleotide exchange factors (GEFs) for Rho family GTPases that activate pathways leading to actin cytoskeletal rearrangements and transcriptional alterations. Its Entrez reference is 7409.

Thus, in some embodiments, the method of the present invention comprises the steps of i) detecting the at least one mutation in a nucleic acid sample obtained from the patient and ii) administering to the patient the therapeutically effective amount of the anti-CD3 antibody when said mutation is detected.

As used herein, the term “nucleic acid sample” refers to any biological sample isolated from the subject liable to contain nucleic acid for the purpose of the present invention. In some embodiments, the sample is a blood sample. The term “blood sample” means any blood sample derived from the patient that contains nucleic acids. Peripheral blood is preferred, and mononuclear cells (PBMCs) are the preferred cells. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art. The template nucleic acid need not be purified. Nucleic acids may be extracted from a sample by routine techniques such as those described in Diagnostic Molecular Microbiology: Principles and Applications (Persing et al. (eds), 1993, American Society for Microbiology, Washington D.C.).

Detecting the mutation may be determined according to any genotyping method known in the art. Typically, common genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, sequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection. Typically, detecting the gain-of-function mutations in the TCR/NF-κB pathway is performed by sequencing, in particular by next generation sequencing. As used herein, the term “next generation sequencing” has its general meaning in the art and refers to sequencing technologies having increased throughput as compared to traditional Sanger- and capillary electrophoresis-based approaches, for example with the ability to generate hundreds of thousands or millions of relatively short sequence reads at a time. Some examples of next generation sequencing techniques include, but are not limited to, sequencing by synthesis, sequencing by ligation, and sequencing by hybridization. A next-generation sequencer can include a number of different sequencers based on different technologies, such as Illumina (Solexa) sequencing, Roche 454 sequencing, Ion torrent sequencing, SOLiD sequencing, and the like.

As used herein, the term “CD3” has its general meaning in the art and refers to to the protein complex associated with the T cell receptor is composed of four distinct chains. In mammals, the complex contains a CD3γ chain, a CD3δ chain, and two CD3ε chains. These chains associate with the TCR and the ζ-chain (zeta-chain) to generate an activation signal in T lymphocytes. The TCR, ζ-chain, and CD3 molecules together constitute the TCR complex.

As used herein, the term “anti-CD3 antibody” include antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). More particularly, the anti-CD3 antibody of the present invention binds to the epsilon (c) chain of the CD3/TCR complex present at the surface of all peripheral T cells According, to the present invention, the anti-CD3 antibody of the present invention is particularly suitable for inducing apoptosis of leukemic cells, in particular leukemic cells that harbor at least one gain-of function mutation as described above. Methods of testing the ability of the anti-CD3 antibody for inducing apoptosis are well known in the art and typically those described in the EXAMPLE.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (VH-CDR1), residues 50-65 (VH-CDR2) and residues 95-102 (VH-CDR3) according to the Kabat numbering system. The CDRs of the light chain variable domain are located at residues 24-34 (VL-CDR1), residues 50-56 (VL-CDR2) and residues 89-97 (VL-CDR3) according to the Kabat numbering system.

As used herein, the term “specificity” refers to the ability of an antibody to detectably bind an epitope presented on an antigen, such as a CD3, while having relatively little detectable reactivity with non-CD3 proteins or structures (such as other proteins presented on T cells, or on other cell types). Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a CD3 polypeptide). The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

In some embodiments, the antibody of the present invention is a chimeric antibody. As used herein, the term “chimeric antibody” refers to an antibody which comprises a VH domain and a VL domain of a non-human antibody, and a CH domain and a CL domain of a human antibody.

In some embodiments, the antibody of the present invention is a humanized antibody. The term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of non-human species (donor antibody) such as mouse, rat or rabbit having the desired specificity, affinity, and capacity.

In some embodiments, the antibody of the present invention is a human antibody. As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). More specifically, the term “human monoclonal antibody”, as used herein, is not intended to include antibodies in which CDR sequences derived from the germline of another mammalian species, such as a mouse, have been grafted onto human framework sequences.

A number of anti-CD3 antibodies are known, including but not limited to, OKT3 (muromonab/Orthoclone OKT3™, Ortho Biotech, Raritan, N.J.; U.S. Pat. No. 4,361,549); hOKT3Y1 (teplizumab) (MGA031) (Herold et al., NEJM 346 (22): 1692-1698 (2002); Foralumab” and/or “28F11, TRX4 (otelixizumab); HuM291 (Nuvion™, Protein Design Labs, Fremont, Calif.); gOKT3-5 (Alegre et al., J. Immunol. 148 (11): 3461-8 (1992); 1F4 (Tanaka et al., J. Immunol. 142: 2791-2795 (1989)); G4.18 (Nicolls et al., Transplantation 55: 459-468 (1993)); 145-2C11 (Davignon et al., J. Immunol. 141 (6): 1848-54 (1988)); and as described in Frenken et al., Transplantation 51 (4): 881-7 (1991); U. S. Pat. Nos. 6,491,9116, 6,406,696, and 6,143,297).

In some embodiments, the anti-CD3 antibody of the present invention comprises a VH and a VL domain selected from Table A.

| Name | VH domain | VL domain |
|---|---|---|
| foralumab | QVQLVESGGGVVQPGRSLRLSCAASGFK | EIVLTQSPATLSLSPGERATLSCRASQS |
| | FSGYGMHWVRQAPGKGLEWVAVIWYDGS | VSSYLAWYQQKPGQAPRLLIYDASNRAT |
| | KKYYVDSVKGRFTISRDNSKNTLYLQMN | GIPARFSGSGSGTDFTLTISSLEPEDFA |
| | SLRAEDTAVYYCARQMGYWHFDLWGRGT | VYYCQQRSNWPPLTFGGGTKVEIK |
| | LVTVSS (SEQ ID NO: 1) | (SEQ ID NO: 2) |
| muromonab | QVQLQQSGAELARPGASVKMSCKASGYT | QIVLTQSPAIMSASPGEKVTMTCSASSS |
| | FTRYTMHWVKQRPGQGLEWIGYINPSRG | VSYMNWYQQKSGTSPKRWIYDTSKLASG |
| | YTNYNQKFKDKATLTTDKSSSTAYMQLS | VPAHFRGSGSGTSYSLTISGMEAEDAAT |
| | SLTSEDSAVYYCARYYDDHYCLDYWGQG | YYCQQWSSNPFTFGSGTKLEIK |
| | TTLTVSS (SEQ ID NO: 3) | (SEQ ID NO: 4) |
| otelixizumab | EVQLLESGGGLVQPGGSLRLSCAASGFT | DIQLTQPNSVSTSLGSTVKLSCTLSSGN |
| | FSSFPMAWVRQAPGKGLEWVSTISTSGG | IENNYVHWYQLYEGRSPTTMIYDDDKRP |
| | RTYYRDSVKGRFTISRDNSKNTLYLQMN | DGVPDRFSGSIDRSSNSAFLTIHNVAIE |
| | SLRAEDTAVYYCAKFRQYSGGFDYWGQG | DEAIYFCHSYVSSFNVFGGGTKLTVL |
| | TLVTVSS (SEQ ID NO: 5) | (SEQ ID NO: 6) |
| teplizumab | QVQLVQSGGGVVQPGRSLRLSCKASGYT | DIQMTQSPSSLSASVGDRVTITCSASSS |
| | FTRYTMHWVRQAPGKGLEWIGYINPSRG | VSYMNWYQQTPGKAPKRWIYDTSKLASG |
| | YTNYNQKVKDRFTISRDNSKNTAFLQMD | VPSRFSGSGSGTDYTFTISSLQPEDIAT |
| | SLRPEDTGVYFCARYYDDHYCLDYWGQG | YYCQQWSSNPFTFGQGTKLQIT |
| | TPVTVSS (SEQ ID NO: 7) | (SEQ ID NO: 8) |
| visilizumab | QVQLVQSGAEVKKPGASVKVSCKASGYT | DIQMTQSPSSLSASVGDRVTITCSASSS |
| | FISYTMHWVRQAPGQGLEWMGYINPRSG | VSYMNWYQQKPGKAPKRLIYDTSKLASG |
| | YTHYNQKLKDKATLTADKSASTAYMELS | VPSRFSGSGSGTDFTLTISSLQPEDFAT |
| | SLRSEDTAVYYCARSAYYDYDGFAYWGQ | YYCQQWSSNPPTFGGGTKVEIK |
| | GTLVTVSS (SEQ ID NO: 9) | (SEQ ID NO: 10) |

In some embodiments, the anti-CD3 antibody of the present invention is muromonab having a light chain as set forth in SEQ ID NO:11 and a heavy chain as set forth in SEQ ID NO:12.

>Muromonab-CD3 light chain
SEQ ID NO: 11
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSG
TKLEINRADTAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSFNRNEC
>Muromonab-CD3 heavy chain
SEQ ID NO: 12
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSAKTTAPSVYPLAPVCGGTTGSSVTLGCLVKG
YFPEPVTLTWNSGSLSSGVHTFPAVLQSDLYTLSSSVTVTSSTWPSQSIT
CNVAHPASSTKVDKKIEPRPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

In some embodiments, the anti-CD3 antibody of the present invention is teplizumab having a light chain as set forth in SEQ ID NO:13 and a heavy chain as set forth in SEQ ID NO:14.

>8869_L|teplizumab|Humanized||L-KAPPA (V-KAPPA(1-
105) + C-KAPPA (107-213)) |||||||213||||
MW 23304.7|MW 23304.7|
SEQ ID NO: 13
DIQMTQSPSSLSASVGDRVTITCSASSSVSYMNWYQQTPGKAPKRWIYDT
SKLASGVPSRFSGSGSGTDYTFTISSLQPEDIATYYCQQWSSNPFTFGQG
TKLQITRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
>8869_H|teplizumab|Humanized||H-GAMMA-1 (VH(1-
119) + CH1(120-217) + HINGE-REGION(218-232) +
CH2(233-342) + CH3(343-449))
|||||||449|||MW 49611.9| MW 49611.9
SEQ ID NO: 14
QVQLVQSGGGVVQPGRSLRLSCKASGYTFTRYTMHWVRQAPGKGLEWIGY
INPSRGYTNYNQKVKDRFTISRDNSKNTAFLQMDSLRPEDTGVYFCARYY
DDHYCLDYWGQGTPVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK

In some embodiments, the anti-CD3 antibody of the present invention is a non-mitogenic anti-CD3 antibody. As used herein, the term “non-mitogenic” has its general meaning in the art and has properties to bind to the CD3 antigen but without inducing cytokine production, as described in U.S. Pat. No. 7,041,289 B1. Thus, a non-mitogenic anti-CD3 antibody delivers for instance a partial T cell signal that renders activated T cells unresponsive. In some embodiments, the non-mitogenic anti-CD3 antibody is otelixizumab or teplizumab.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumour size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the anti-CD3 antibody of the present invention is administered to the subject in the form of a pharmaceutical composition, which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, the anti-CD3 antibody of the present invention is administered to the patient in combination with chemotherapy. As used herein, the term “chemotherapy” has its general meaning in the art and refers to the treatment that consists in administering to the patient a chemotherapeutic agent. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine;

acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

The invention will be further illustrated by the following figures and examples. However, these examples and figures should not be interpreted in any way as limiting the scope of the present invention.

### FIGURES

FIG. 1: (a) TCR-pathway/NFKB mutated patients showed poorer outcome as compared to unmutated cases (b) investigation of the effects of OKT3 exposure on 4 ATL samples including 2 cases harboring CARD11 and PRKCB gain of function alterations and 2 cases without any TCR pathway mutation. These preliminary data suggest that ATL harboring TCR pathway mutations clearly responded to anti-CD3.

### EXAMPLE

We performed an integrated genomic analysis of a retrospective cohort of 62 ATL patients mainly originating from Africa and the Caribbean area. This task was achieved by targeted deep sequencing, SNP array analysis, RNA sequencing and high throughput sequencing (HTS) based mapping of proviral integration sites. The genomic landscape in ATL from these populations overlaps with slight modifications with the genomic alterations observed in ATL from Japan (Kataoka K et al (2015) Nat Genet 47:1304-1315). Interestingly, a subset of mutations (e.g. activating mutations in the NOTCH and JAK/STAT pathways) is shared with T-ALL. Moreover, most of these genetic alterations were not restricted to ATL lymphomagenesis but are common to other non-viral peripheral T-cell lymphomas. Genomic alterations found in ATL were clustered in three main pathways: 46 (74%) patients harbored alterations affecting the TCR/NF-κB pathway (PLCG1, CARD11, PRKCB, CBLB, IRF4, CSNK1A1, FYN, RHOA, VAV1); 26 (42%) harbored alterations (mutations and deletions) affecting T-cell trafficking (CCR4, CCR7, GP183) and 20 (32%) showed alterations in gene involved in immune escape (FAS, HLA-B, B2M, CD58). Importantly, FAS mutations (10/62) predominated in aggressive cases and are identical to germline mutations observed in patients with autoimmune lymphoproliferative syndrome (ALPS), leading to impaired T-cell activation induced cell death (AICD).

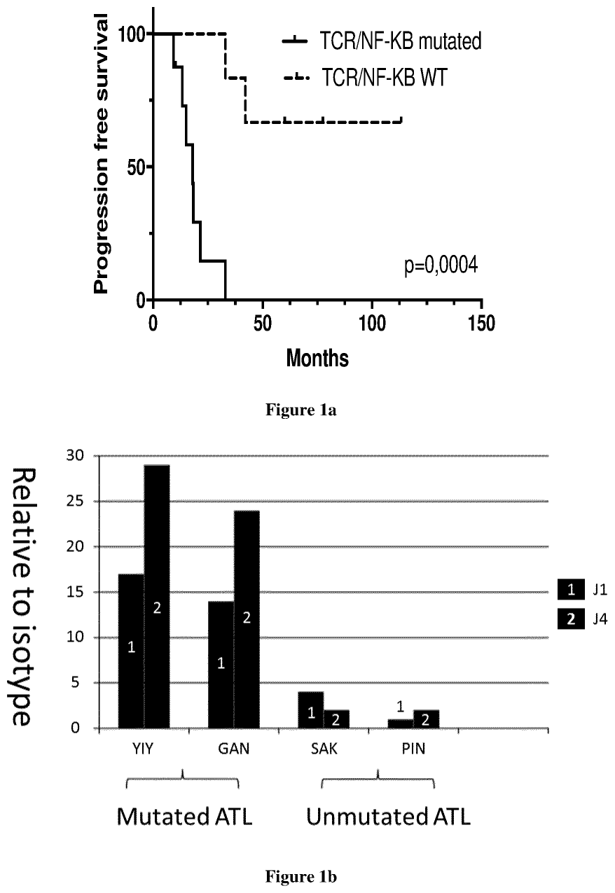

We also developed in collaboration with the GIGA Institute in Liege a protocol of linker-mediated PCR (LM-PCR) followed by high-throughput sequencing (HTS) that is used to map and quantify unique HTLV-1 proviral integration sites. This technique allows us to monitor tumor viral clones during disease course and can be used as a minimal residual disease detection tool (Artesi M et al (2017) Leukemia 31:2532-2535; Rosewick N et al (2017) Nat Comm 8:15264). Under our previously described in vitro conditions (Trinquand A, dos Santos N, Tran Quang C et al (2016) Cancer Discov 6:972-85), we investigated the effects of OKT3 exposure on 4 ATL samples including 2 cases harboring CARD11 and PRKCB gain of function alterations and 2 cases without any TCR pathway mutation. Our data suggest that ATL harboring TCR pathway mutations clearly responded to anti-CD3 (FIG. 1B, red+OKT3) and died by apoptosis possibly by a mechanism resembling AICD. Importantly, these TCR-pathway/NFKB mutated patients also showed poorer outcome as compared to unmutated cases (FIG. 1A).

### REFERENCES

Throughout this application, various references describe the state of the art to which this invention pertains. The disclosures of these references are hereby incorporated by reference into the present disclosure.

1. A method of treating adult T-cell leukemia/lymphoma (ATL) in a patient in need thereof comprising administering to the patient a therapeutically effective amount of an anti-CD3 antibody.

2. The method of claim 1 wherein the patient harbors at least one gain-of-function mutation in a gene involved in the TCR/NF-κB pathway.

3. The method of claim 1 wherein the patient harbors at least one gain-of-function mutation in PLCG1, CARD11, PRKCB, CBLB, IRF4, CSNK1A1, FYN, RHOA, or VAV1.

4. The method of claim 1 further comprising the steps of i) detecting the at least one gain-of-function mutation in a nucleic acid sample obtained from the patient and ii) administering to the patient the therapeutically effective amount of the anti-CD3 antibody when said at least one gain-of-function mutation is detected.

5. The method of claim 1 wherein the anti-CD3 antibody is a chimeric antibody, a humanized antibody or a human antibody.

6. The method of claim 1 wherein the anti-CD3 antibody is selected from the group consisting of foralumab, muromonab, otelixizumab, teplizumab and visilizumab.

7. The method of claim 1 wherein the anti-CD3 antibody is muromonab having a light chain as set forth in SEQ ID NO:11 and a heavy chain as set forth in SEQ ID NO:12.

8. The method of claim 1 wherein the anti-CD3 antibody is teplizumab having a light chain as set forth in SEQ ID NO:13 and a heavy chain as set forth in SEQ ID NO:14.

9. The method of claim 1 wherein the anti-CD3 antibody is a non-mitogenic anti-CD3 antibody.

10. The method of claim 1 wherein the anti-CD3 antibody is administered to the patient in combination with chemotherapy.

11. The method of claim 3, wherein the at least one gain-of-function mutation in PLCG1 is S345F, Q718K, DelEF730, G869F, S739T, Q916E, E1163K, R48W, D1165H, D1165E or DelDQ1169.

12. The method of claim 3, wherein the at least one gain-of-function mutation in CARD11 is D401N, R179W, R337Q, D401N, R423W, D357V, R377Q, R707C or E626.

13. The method of claim 3, wherein the at least one gain-of-function mutation in PRKCB is D427N, Q433K, A25V or D470H.

14. The method of claim 3, wherein the at least one gain-of-function mutation in CBLB is InsGH296.

15. The method of claim 3, wherein the at least one gain-of-function mutation in IRF4 is K59R, L70V, L64L, E109Q, S11R.

16. The method of claim 3, wherein the at least one gain-of-function mutation in CSNK1A1 is S189R or L160F.

17. The method of claim 3, wherein the at least one gain-of-function mutation in FYN is T15K or R206C).

18. The method of claim 3, wherein the at least one gain-of-function mutation in RHOA is C16Y, C16R, G17V, G124S, D120N, D120V, A161P or A161V.

19. The method of claim 3, wherein the at least one gain-of-function mutation in VAV1 is F69V, L145P, R195C, Q498K, M501L or N505T.
